(19) United States
(12) Patent Application Publication
(10) Pub. No.: US 2024/0029205 A1
(43) Pub. Date: Jan. 25, 2024

(54) SYSTEMS AND METHODS FOR MULTI-SOURCE MEDICAL IMAGING RECONSTRUCTION

(71) Applicant: Boston Scientific Scimed, Inc., Maple Grove, MN (US)

(72) Inventors: Kyle TRUE, Minneapolis, MN (US); Daniel J. FOSTER, Lino Lakes, MN (US); Sebastian ORDAS CARBONI, Vadnais Heights, MN (US)

(73) Assignee: Boston Scientific Scimed, Inc., Maple Grove, MN (US)

(21) Appl. No.: 18/350,818

(22) Filed: Jul. 12, 2023

Related U.S. Application Data
(60) Provisional application No. 63/368,840, filed on Jul. 19, 2022.

(51) Int. Cl. G06T 5/50; G06T 7/00

(52) U.S. Cl. CPC G06T 5/50; G06T 7/0014; G06T 2207/10081; G06T 2207/10136; G06T 2207/10116

(57) ABSTRACT

Medical imaging reconstruction of a subject's anatomy may include: monitoring first and second medical imaging data of the subject's anatomy, wherein: the first medical imaging data is captured from within the subject's anatomy by at least one first medical imaging device; the second medical imaging data is captured from outside of the subject's anatomy by at least one second medical imaging device; and at least one of the first medical imaging device or the second medical imaging device is operable to selectively target different regions of the subject's anatomy; and in response to detecting, based on the monitoring, a divergence in the first medical imaging data and the second medical imaging data above a predetermined threshold, updating a medical imaging reconstruction of the subject's anatomy that is based on the first medical imaging data and the second medical imaging data.

### CROSS-REFERENCE TO RELATED APPLICATIONS

This application claims the benefit of priority of U.S. Provisional Application No. 63/368,840, filed Jul. 19, 2022, which is incorporated by reference herein in its entirety.

### TECHNICAL FIELD

Various embodiments of this disclosure relate generally to multi-source medical imaging reconstruction, and, more particularly, to systems and methods that account for data recency for medical imaging acquisition and/or reconstruction.

### BACKGROUND

In certain medical procedures, it may be beneficial or even necessary to have a current and accurate understanding of a subject's anatomy and/or the position of a medical device interacting with the patient's internal anatomy. For example, organs or tissue may be shifted during the course of a procedure, an implantable device may require a very high level of accuracy in positioning, the medical device may require a high level of accuracy for targeting (e.g., for an ablation procedure), a practitioner may need to revisit previously imaged structure, or the like. Medical imaging has been used to model or map portions of the subject's anatomy, e.g., prior to and/or during a medical procedure. In some instances, a single medical imaging device may have a field of view that is too narrow to capture a full scope of relevant anatomy in some types of procedures. To expand the field of view, inside and outside imaging has been used, in which a first source of medical imaging within the subject is fused with a second source of medical imaging captured externally to the subject in order to generate a combined reconstruction modelling the subject's anatomy.

However, an inherent challenge with the fusion of different sources of medical imaging with inside and outside imaging is that some information used for the reconstruction is older than other information. In situations in which tissue or organs may shift during a procedure, older information may be a less valid representation of reality. An operator may not be able to discern which parts of the reconstruction are valid and which are not, and thus may also not be able to judge which portions of the subject's anatomy need to be reimaged, or when. This type of loss in validity, fidelity, or certainty may increase the time needed for a procedure, decrease accuracy for the medical device, increase risk of harm to the subject, increase a cost or complexity of the procedure, or the like.

This disclosure is directed to addressing above-referenced challenges. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, methods and systems are disclosed for providing in vivo navigation of a medical device.

In one aspect, a computer-implemented method for medical imaging reconstruction of a subject's anatomy may include: monitoring first medical imaging data of the subject's anatomy and second medical imaging data of the subject's anatomy, wherein: the first medical imaging data is captured from within the subject's anatomy by at least one first medical imaging device; the second medical imaging data is captured from outside of the subject's anatomy by at least one second medical imaging device; and at least one of the first medical imaging device or the second medical imaging device is operable to selectively target different regions of the subject's anatomy; and in response to detecting, based on the monitoring, a divergence in the first medical imaging data and the second medical imaging data above a predetermined threshold, updating a medical imaging reconstruction of the subject's anatomy that is based on the first medical imaging data and the second medical imaging data.

In one aspect, updating the medical imaging reconstruction of the subject's anatomy may include: causing the at least one of the first medical imaging device or the second medical imaging device to acquire first or second medical imaging data, respectively, from at least one region of the subject's anatomy such that the acquisition causes a reduction in the divergence; and modifying the medical imaging reconstruction of the subject's anatomy based on the acquired first or second medical imaging data.

In one aspect, causing the at least one of the first medical imaging device or the second medical imaging device to reacquire first or second medical imaging data, respectively, may include operating an actuator configured to one or more of reposition or reorient the at least one of the first medical imaging device or the second medical imaging device so that the at least one of the first medical imaging device or the second medical imaging device is targeting the at least one region of the subject's anatomy.

In one aspect: the at least one region of the subject's anatomy may include a plurality of regions; and the actuator may be operated so that the first or second medical imaging data is reacquired for the plurality of regions according to one or more of a random or pseudo-random pattern, a linear pattern, a tilt pattern, a rotational pattern, or an obstacle-avoidance protocol based on a signal from a force transducer associated with the actuator.

In one aspect: the at least one of the first medical imaging device or the second medical imaging device that is operable to selectively target different regions of the subject's anatomy may include a plurality of sensors or sensor arrays, each of the plurality of sensors or sensor arrays one or more of positions or oriented so as to target different regions of the subject's anatomy; and causing the at least one of the first medical imaging device and the second medical imaging device to reacquire first or second medical imaging data, respectively, may include changing a targeting of the plurality of sensors or sensor arrays.

In one aspect, the divergence in the first medical imaging data and the second medical imaging data may be based on a distance between one or more of: a first region of the subject's anatomy targeted by the first medical imaging device and a second region of the subject's anatomy targeted by the second medical imaging device; or a first location of the first medical imaging device and a second location of the second medical imaging device.

In one aspect, the divergence in the first medical imaging data and the second medical imaging data may be based on a moving average of the distance.

In one aspect, the method may further include one or more of: determining one or more of the first location of the first medical imaging device or the first region of the subject's anatomy targeted by the first medical imaging device based on one or more of electromagnetic position tracking, fiber optic shape sensing, location tracking via the second medical imaging device, or impedance-based tracking: or determining one or more of the second location of the second medical imaging device or the first region of the subject's anatomy targeted by the second medical imaging device based on one or more of electromagnetic position tracking, optical tracking, or internal position tracking of the second medical imaging device.

In one aspect, the divergence in the first medical imaging data and the second medical imaging data may be based on an extent of time elapsed since (a) one or more of the first medical imaging data was captured by the first medical imaging device or (b) the second medical imaging data was captured by the second medical imaging device.

In one aspect, the method may further include: generating the medical imaging reconstruction of the subject's anatomy based on the first medical imaging data and the second medical imaging data; and causing a display to output a visual depiction of at least a portion of the medical imaging reconstruction.

In one aspect, updating the medical imaging reconstruction of the subject's anatomy may include modifying the visual depiction of the at least portion of the medical imaging reconstruction such that at least a portion of the medical imaging reconstruction has a visual appearance indicative of an extent of time elapsed since medical imaging data upon which the at least portion is based was captured.

In one aspect, the visual appearance indicative of an extent of time elapsed since medical imaging data upon which the at least portion is based was captured may include one or more of: a reduction in opacity, contrast, saturation, sharpness, resolution, brightness, or combinations thereof, in which a magnitude of the reduction increases with an increase in the extent of time elapsed; a shift in color, in which a magnitude of the shift increases with an increase in the extent of time elapsed; a flashing effect; or a bounding region or selection indicating the at least portion of the medical imaging reconstruction.

In one aspect, the method may further include: obtaining a prior medical imaging reconstruction of the subject's anatomy based on third medical imaging data captured by a third medical imaging device; determining a registration of the medical imaging reconstruction with the prior medical imaging reconstruction; and causing the display to output a visual depiction of the prior medical imaging reconstruction in conjunction with the at least portion of the medical imaging reconstruction based on the determined registration.

In one aspect, the method may further include: receiving a user selection for a relative visibility of the prior medical imaging reconstruction and the at least portion of the medical imaging reconstruction in the visual depiction; and selectively increasing or decreasing a visibility of the prior medical imaging reconstruction and a visibility of the at least portion of the medical imaging reconstruction in the visual depiction, respectively, based on the user selection.

In one aspect, the first medical imaging device may include one or more of an ultrasound transducer, a parallel or phased array transducer, an optical coherence tomography device, or an optical sensor.

In one aspect, the second medical imaging device may include one or more of an ultrasound transducer, an array of ultrasound transducers, an X-ray device, or a computed tomography device.

In one aspect, a system for medical imaging reconstruction of a subject's anatomy may include: a memory storing instructions; a first medical imaging device configured to capture first medical imaging data from within the subject's anatomy; a second medical imaging device configured to capture second medical imaging data from outside of the subject's anatomy, wherein at least one of the first medical imaging device or the second medical imaging device is operable to selectively target different regions of the subject's anatomy; and a processor that is operatively connected to the memory, the first medical imaging device, and the second medical imaging device, and that is configured to execute the instructions to perform operations. The operations may include: monitoring first medical imaging data of the subject's anatomy and second medical imaging data of the subject's anatomy; and in response to detecting, based on the monitoring, a divergence in the first medical imaging data and the second medical imaging data above a predetermined threshold, updating a medical imaging reconstruction of the subject's anatomy that is based on the first medical imaging data and the second medical imaging data.

In one aspect, updating the medical imaging reconstruction of the subject's anatomy may include: causing the at least one of the first medical imaging device and the second medical imaging device to acquire first or second medical imaging data, respectively, from at least one region of the subject's anatomy that causes a reduction in the divergence; and modifying the medical imaging reconstruction of the subject's anatomy based on the acquired first or second medical imaging data.

In one aspect, the operations may further include: generating the medical imaging reconstruction of the subject's anatomy based on the first medical imaging data and the second medical imaging data; and causing a display to output a visual depiction of at least a portion of the medical imaging reconstruction; wherein updating the medical imaging reconstruction of the subject's anatomy includes modifying the visual depiction of the at least portion of the medical imaging reconstruction such that at least a portion of the medical imaging reconstruction has a visual appearance indicative of an extent of time elapsed since medical imaging data upon which the at least portion is based was captured.

In one aspect, a computer-implemented method for medical imaging reconstruction of a subject's anatomy may include: monitoring first medical imaging data of the subject's anatomy and second medical imaging data of the subject's anatomy, wherein: the first medical imaging data is captured from within the subject's anatomy by at least one first medical imaging device; the second medical imaging data is captured from outside of the subject's anatomy by at least one second medical imaging device; and at least one of the first medical imaging device or the second medical imaging device is operable to selectively target different regions of the subject's anatomy; and in response to detecting, based on the monitoring, a divergence in the first medical imaging data and the second medical imaging data above a predetermined threshold, updating a medical imaging reconstruction of the subject's anatomy that is based on the first medical imaging data and the second medical imaging data, wherein updating the medical imaging reconstruction of the subject's anatomy includes: causing the at least one of the first medical imaging device or the second medical imaging device to acquire first or second medical imaging data, respectively, from at least one region of the subject's anatomy such that the acquisition causes a reduction in the divergence; modifying the medical imaging reconstruction of the subject's anatomy based on the acquired first or second medical imaging data; and modifying the visual depiction of the at least portion of the medical imaging reconstruction such that at least a portion of the medical imaging reconstruction has a visual appearance indicative of an extent of time elapsed since medical imaging data upon which the at least portion is based was captured.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and, together with the description, serve to explain the principles of the disclosed embodiments.

FIG. 1 depicts an exemplary environment for generating a medical imaging reconstruction, according to one or more embodiments.

FIG. 2 depicts an exemplary embodiment of a first medical imaging device integrated into a medical device, according to one or more embodiments.

FIG. 3 depicts an exemplary embodiment of a second medical imaging device.

FIG. 4 is a flow diagram of an exemplary process for generating a medical imaging reconstruction, according to one or more embodiments.

FIG. 5 depicts an exemplary embodiment of a medical imaging reconstruction.

FIGS. 6A and 6B depict schematic illustrations of using a medical imaging device within anatomy of a subject.

FIG. 6C depicts an exemplary embodiment of a visual representation of medical imaging data taken in the illustration of FIG. 6B.

FIG. 7 depicts an exemplary embodiment of a graphical user interface for a visual depiction of a medical imaging reconstruction, according to one or more embodiments.

FIG. 8 depicts an exemplary embodiment of a graphical user interface for a visual depiction of an instance of medical imaging data used to form the medical imaging reconstruction in the graphical user interface of FIG. 7.

FIG. 9 depicts another embodiment of a graphical user interface for a visual depiction of a medical imaging reconstruction, according to one or more embodiments.

FIG. 10 depicts another exemplary embodiment of a medical imaging reconstruction.

FIG. 11 depicts an exemplary graphical user interface for adjusting visibility of different potions of a medical imaging reconstruction.

FIG. 12 depicts an example of a computing device, according to one or more embodiments.

### DETAILED DESCRIPTION OF EMBODIMENTS

According to certain aspects of the disclosure, methods and systems are disclosed for multi-source medical imaging reconstruction, and, more particularly, to systems and methods that account for data recency for medical imaging acquisition and/or reconstruction. As used herein, accounting for “data recency” generally encompasses accounting for how much time has elapsed since data was captured, and may be applied to various activities such as procedures for further acquisition of data, how data is displayed or modelled, etc.

In certain medical procedures, it may be desirable or necessary to have an accurate representation of the anatomy of a subject. For example, in ablation procedures, accurate knowledge of location and orientation of an ablation device within the subject's anatomy, as well as accurate knowledge of locations of various structures of the subject's anatomy, in particular tissue to be ablated, may facilitate a successful outcome. However, conventional navigation techniques may not be suitable. For example, conventional techniques may not account for data recency, and thus may not provide an accurate representation of the positions of structures of the subject's anatomy or the location of a medical device within the subject. As will be discussed in more detail below, in various embodiments, systems and methods are described for medical imaging reconstruction that account for data recency.

Reference to any particular procedure is provided in this disclosure only for convenience and not intended to limit the disclosure. Examples of procedures that may be relevant to this disclosure include, for example, a resection or removal of a lesion, a dilation, a retrieval, a biopsy, an implantation, an ablation, delivery of a therapeutic agent, etc. A person of ordinary skill in the art would recognize that the concepts underlying the disclosed devices and methods may be utilized in any suitable procedure. The disclosure may be understood with reference to the following description and the appended drawings, wherein like elements are referred to with the same reference numerals.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

For ease of description, portions of the device and/or its components are referred to as proximal and distal portions. It should be noted that the term “proximal” is intended to refer to portions closer to a user of the device, and the term “distal” is used herein to refer to portions further away from the user. Similarly, extends “distally” indicates that a component extends in a distal direction, and extends “proximally” indicates that a component extends in a proximal direction.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. The term “or” is used disjunctively, such that “at least one of A or B” includes, (A), (B), (A and A), (A and B), etc. Relative terms, such as, “substantially” and “generally,” are used to indicate a possible variation of ±10% of a stated or understood value.

As used herein, terms such as “medical imaging data” or the like generally encompass data associated with and/or indicative of a geometry and/or physiology of a subject, e.g., that may be generating via medical imaging. Medical imaging generally encompasses techniques whereby a signal (light, electromagnetic energy, radiation, etc.) is generated, and measurements are taken that are indicative of how that signal interacts with and/or is affected by, transmitted through, or the like, the subject. Examples of medical imaging technologies include Ultrasound scans, Magnetic Resonance Imaging (MRI) scans, Computed Tomography (CT) scans, X-ray scans, visual imaging (e.g., via an optical channel or camera or the like), or any other suitable modality, e.g., that may be used to visualize an interior of at least a portion of the subject's anatomy. Medical imaging data may include, for example, two-dimensional data and/or images, three-dimensional data and/or images, voxel or pixel data, and/or any other suitable data associated with the subject and/or medical imaging.

As used herein, a “medical imaging reconstruction” generally encompasses a model, image, visual representation, or the like generated using medical imaging data. For example, a reconstruction may include a visual depiction of a two-dimensional or three-dimensional geometric model of at least a portion of subject anatomy generated based on medical imaging data, a video, a time-varying image, a solid model of the portion of subject anatomy, a mesh of nodes or points representative of the portion of the anatomy, and/or any other suitable characteristics of the portion of the anatomy that may be represented visually, as a mechanical or physiological model, or the like.

As used herein, the term “user” or the like generally encompasses any person or entity that may interact with or operate a device or component. An “operator” generally encompasses any personnel involved in providing medical care such as, for example, a doctor, a surgeon, a nurse, a radiologist, a technologist, a hospital, doctor's office, or outpatient facility, or the like. A “subject” generally encompasses a person receiving such medical care.

For many interventional procedures, e.g., procedures which involve interacting with or navigating a medical device within the anatomy of a subject, the subject may receive pre-procedure imaging, e.g., an MRI scan, a CT scan, X-Ray scan, or the like, which may be used by a medical provider, such as a physician, surgeon, or the like, to plan the procedure. In some cases, prior imaging results and/or reconstructions generated therefrom may be available during the procedure, e.g., to act as a reference or guide.

However, prior imaging may not be a valid reflection of reality for the subject's anatomy. For example, from the time at which prior imaging was taken to a time when a procedure is performed, or even during the procedure itself, organs or tissue may shift, regions of diseased tissue may progress, etc. Various other factors, such as subject hydration, diet, or the like, may also cause changes to the subject's anatomy. Thus, in some cases, prior imaging may be supplemented with further imaging captured during the procedure. For example, scope devices, such as a ureteroscopes, duodenoscopes, gastroscopes, endoscopic ultrasonography (“EUS”) scopes, colonoscopes, bronchoscopes, laparoscopes, arthroscopes, cystoscopes, aspiration scopes, sheaths, catheters, or the like, may include one or more optical channel(s), one or more image sensor(s), one or more ultrasound transducer(s), or the like that may provide medical imaging during an interventional procedure. However, medical imaging captured using a device within the subject's anatomy may have a narrow field of view, and may not be suitable for achieving a full understanding of the portion of the subject's anatomy relevant to the procedure.

Medical imaging captured externally from the subject has also been used during a procedure, e.g., X-Ray, percutaneous ultrasound, CT, etc. However, such techniques, although generally having a larger field of view than internal imaging, may not have sufficient accuracy for certain procedures.

And, while a combination of internal medical imaging and external medical imaging, e.g., inside and outside imaging, have been used during medical procedures, such combination also generally presents challenges with regard to accuracy and validity. For example, an inherent challenge with the fusion of different sources of medical imaging with inside and outside imaging is that some information used for the reconstruction is older than other information. Because, as discussed above, tissue or organs may shift between imaging being captured and a procedure being performed or during a procedure, older information may be a less valid representation of reality. In other words, some portions of the reconstruction may be less valid than others, and it may not be possible for the medical provider to distinguish which parts of a reconstruction are reliable, and which are not. The operator may also have no way of knowing an appropriate time to reacquire medical imaging data. As a result, inside and outside imaging may lead to an inherent risk of the operator relying on invalid data.

The foregoing difficulties are exacerbated when, for example, the procedure and/or the medical device being used is sensitive to minor changes in the subject's anatomy. Without a clear indication as to the validity of any particular medical imaging relative to a current state of the subject's anatomy, the medical provider may rely on inaccurate medical imaging data, which may negatively impact the procedure or the subject's outcome. Accordingly, improvements in technology relating to accurately reflecting the validity of medical imaging reconstructions are needed.

In the following description, embodiments will be described with reference to the accompanying drawings. As will be discussed in more detail below, in various embodiments, systems and methods for providing in vivo navigation of a medical device are described.

In an exemplary use case, a subject may receive pre-procedure medical imaging. During a procedure, the subject may receive inside and outside imaging. For example, a first medical imaging device, such as an ultrasound transducer, may be included on a medical device inserted into the subject's anatomy (e.g., an endoscope), and a second medical imaging device, e.g., a transabdominal or transthoracic ultrasound sensor, may be used for external imaging. One or more of first medical imaging data from the first medical imaging device, second medical imaging data from the second medical imaging device, or pre-procedure medical imaging data may be registered with each other and/or used to generate a medical imaging reconstruction of the subject's anatomy, e.g., via a medical imaging reconstruction system or the like.

As the procedure is performed, various circumstances may change with regard to the first and second medical imaging data captured by the first and second medical imaging devices. For example, as time passes, a particular instance of medical imaging data captured by a medical imaging device may become less valid. In another example, one of the medical imaging devices may be moved to a different location and/or may target a different region within the anatomy of the subject. Such circumstances may result in a divergence, e.g., a change in state from the period of time at which the medical imaging data was captured. The reconstruction system may monitor the first and second medical imaging data captured by the first and second medical imaging devices, e.g. to determine a divergence in the first and second medical imaging data.

A divergence may be based on or associated with, for example, on one or more of a time elapsed since medical imaging data was captured, a distance or change in distance between the first and second medical devices or a region of the subject's anatomy targeted by the first and second medical imaging devices, respectively, a registration error between the first and second medical imaging data, or the like. In another example, a divergence may be based on a user selection of a region of the subject's anatomy for imaging that is different than a region currently or previously imaged.

In response to the divergence being above a predetermined threshold, the reconstruction system may cause the medical imaging reconstruction of the subject to be updated. Such updating may include, for example, causing one or more of the first or second medical imaging devices to reacquire medical imaging data and modifying the medical imaging reconstruction based on the reacquired data, or modifying a visual depiction of the medical imaging reconstruction such that at least a portion of the medical imaging reconstruction has a visual appearance indicative of an extent of time elapsed since medical imaging data upon which the at least portion is based was captured.

In an example, the first medical imaging device may be moved within the subject's anatomy. As a result of such motion, the first medical imaging device may target different regions of the subject's anatomy, and may move relative to the second medical imaging device. As the first medical imaging device moves, captured first medical imaging data may be used to update the medical imaging reconstruction. Further, as time passes after previous regions of the subject's anatomy were targeted, corresponding portions of the medical imaging reconstruction may be grayed out, blurred, or the like to visually indicate the age of the data used as a basis for those portions. Additionally, the age of data for a previously targeted region and/or the increasing distance between the first medical imaging device and the second medical imaging device may cause the registration system to trigger a reacquisition of data by the second medical imaging device.

The examples above are intended to be illustrative only, and it should be understood that techniques according to this disclosure may be adapted to any suitable type of procedure or activity, and that further aspects of this disclosure are discussed in further detail below. Further, while some of the examples above pertained to ultrasound imaging, it should be understood that any suitable type of medical imaging may be used.

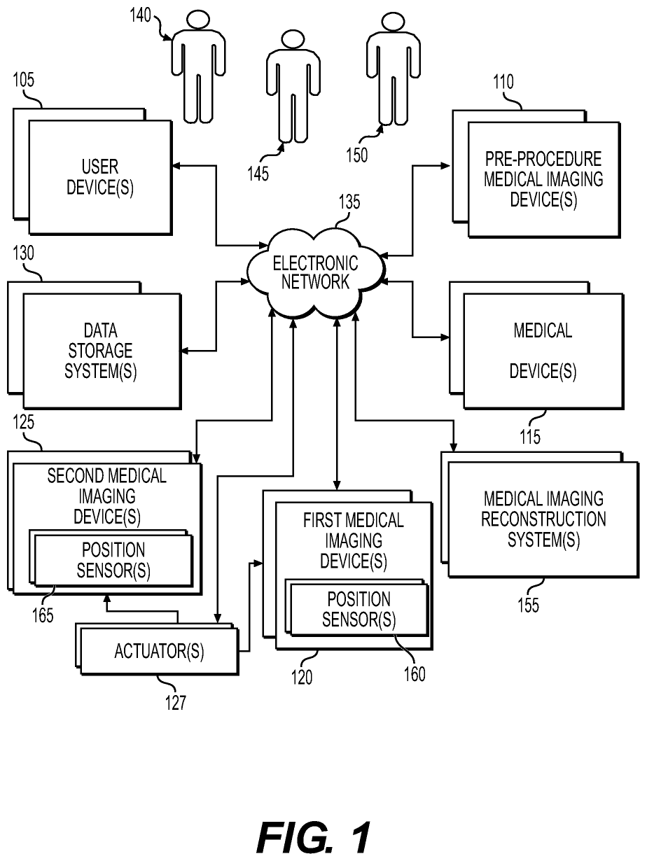

FIG. 1 depicts an exemplary environment that may be utilized with techniques presented herein. One or more user device(s) 105, one or more pre-procedure medical imaging device(s) 110, one or more medical device(s) 115, one or more first medical imaging device(s) 120 for inside imaging, one or more second medical imaging device(s) 125 for outside imaging, one or more actuator(s) 127, and one or more data storage system(s) 130 may communicate across an electronic network 135. One or more user(s) 140 may interact with the user device 105, e.g., in order to interact with and/or access information from another component of the environment. One or more operator(s) 145 may use, access, or direct the use of one or more of the components in the environment, e.g., in order to provide a medical service for a subject 150, e.g., a medical intervention, a medical procedure, a diagnosis, etc. As will be discussed in further detail below, the environment may further include one or more medical imaging reconstruction system(s) 155, e.g., that account for data recency when generating medical imaging reconstructions of anatomy of the subject 150.

In some embodiments, the components of the environment are associated with a common entity, e.g., a hospital, facility, or the like. In some embodiments, one or more of the components of the environment is associated with a different entity than another. The systems and devices of the environment may communicate in any arrangement. As will be discussed herein, systems and/or devices of the environment may communicate and/or operate in order to one or more of perform a medical procedure on the subject 150, or generate a medical imaging reconstruction of the anatomy of the subject 150 that accounts for data recency, among other activities.

The user device 105 may be a computer system such as, for example, a desktop computer, a mobile device, a tablet, etc. In some embodiments, the user device 105 may include one or more electronic application(s), e.g., a program, plugin, browser extension, etc., installed on a memory of the user device 105. In some embodiments, the electronic application(s) may be associated with one or more of the other components in the environment. For example, the electronic application(s) may include software or an application for accessing, controlling, and/or operating a medical device, a medical imaging device, for retrieving or uploading data to a data storage system, for outputting data such as medical imaging reconstructions, or the like. The user device 105 may include any suitable input and output devices (not shown), e.g., a mouse, a keyboard, a microphone, a touch screen, a display, a speaker, etc.

The pre-procedure medical imaging device 110 may be any suitable type of medical imaging device, and may include a similar or different modality of medical imaging as the first and second medical imaging devices 120 and 125. In an example, the pre-procedure medical imaging device 110 may include a modality of medical imaging device that may be less easy or impossible to use during the course of a medical procedure. In a non-limiting example, MRI scanners may be used to generate a relatively detailed medical imaging reconstruction of subject anatomy, and may have an expanded field of view relative to other medical imaging modalities. However, an MRI scanner may be difficult to efficiently or effectively use during the course of a medical procedure. Not only may the MRI scanner itself obstruct access to the subject 150, but also the medical procedure itself, e.g., movement of the subject 150, the presence of the operator 145 and/or the medical device 115, etc. may interfere with or negatively impact the ability of the MRI scanner to produce accurate or even usable results.

As will be discussed in further detail below, the pre-procedure medical imaging device 110 may be used, for example, to generate an initial medical imaging reconstruction of the subject's anatomy that may act as a guide and/or be combined with additional medical imaging data captured during a medical procedure. Medical imaging data and/or data based thereon such as the initial medical imaging reconstruction may be stored on the data storage system 130.

The medical device 115 may be any suitable device that may be used during a medical procedure, e.g., for the purpose of investigation, diagnosis, treatment, excision, or the like. In one example, a medical device 115 includes a scope device, e.g., an endoscope or the like, that may have a functionalized end, e.g., an ablation tool, a biopsy tool, an optical channel, etc. and/or a working channel for receiving a medical instrument, such as the tools above. In another example, a medical device 115 includes a syringe with a needle configured to deliver and/or obtain fluid within the anatomy of the subject. In alternatives, the medical device 115 may include another type of medical device, such as a laparoscopic instrument or interventional radiology tool. In various embodiments, the medical device 115 may be operated by the operator 145, and/or may be at least partially autonomous. In some embodiments, the medical device 115 may further include a position sensor (not shown) for tracking a position of the medical device 115.

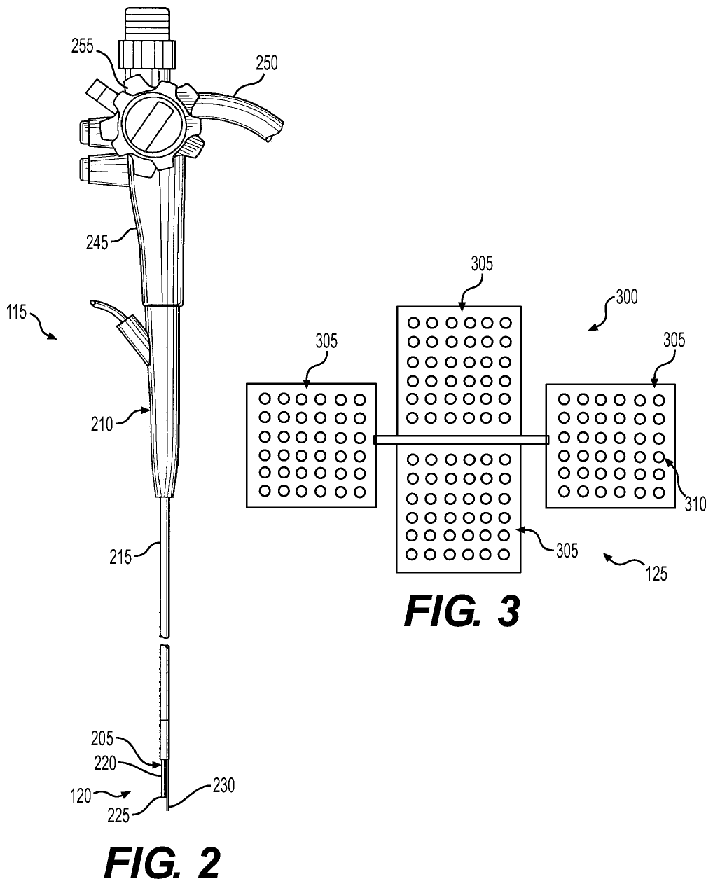

The first medical imaging device 120 is configured to capture first medical imaging data from within the anatomy of the subject 150. The first medical imaging data may be stored by the data storage system 130. In some embodiments, as shown in FIG. 2 and described below, the first medical imaging device 120 may be at least partially integrated into the medical device 115. For example, the first medical imaging device 120 may include an ultrasound transducer at a distal end of a shaft of a scope device of the medical device 115. In other examples, the first medical imaging device 120 and the medical device 115 may be separate devices.

The first medical imaging device 120 may be selectively navigable or orientable within the anatomy of the subject. For example, in examples in which first medical imaging device 120 is integrated into medical device 115, as discussed below, a handle portion 245 (FIG. 2) may control or steer a distal portion of a shaft 215, and the first medical imaging device 120 may be disposed at a distal end portion of the shaft 215. In other examples, in which the first medical imaging device 120 is separate from medical device 115, the first medical imaging device 120 may include an insertion portion for insertion into the anatomy of the subject (e.g., a body lumen of the subject). The first medical imaging device 120 may include an ultrasound transducer at a distal end of the insertion portion. The first medical imaging device 120 may further include a control portion (e.g., a handle) proximal of the insertion portion, which may include actuators or other mechanisms outside of the subject 150, and the control portion may be operable to control or guide the location and/or orientation of the ultrasound transducer within the anatomy of the subject. By adjusting the location and/or orientation of the first medical imaging device 120 within the anatomy of the subject, it may be possible for the first medical imaging device 120 to selectively target different regions of the anatomy of the subject 150.

In some embodiments, the first medical imaging device 120 may include one or more position sensor(s) 160. The position sensor 160 may be configured generate a signal indicative of a location or orientation of the first medical imaging device 120. Such signal may also be used to determine what region of the anatomy of the subject 150 is targeted by the first medical imaging device 120. Any suitable type of position sensor may be used. For example, in an exemplary embodiment, the position sensor 160 may include an electromagnetic position sensor positioned at the distal end of the first medical imaging device 120. In another exemplary embodiment, the first medical imaging device 120 may include a fiber optic channel running from the distal end to the proximal end, and the position sensor 160 may be configured to use fiber optic shape sensing in order to determine an orientation and location of the distal end. In a further embodiment, the second medical imaging device 125 may be usable to determine a location and/or orientation of the first medical imaging device 120, and thus may act as a position sensor.

In another exemplary embodiment, the first medical imaging device 120 may include a plurality of sensors or a plurality of sensor arrays. For example, the first medical imaging device 120 may include an ultrasound array transducer, whereby operating different portions of the ultrasound array transducer enables targeting different regions of the anatomy of the subject 150. In a further exemplary embodiment, the first medical imaging device 120 may not include a portion external to the anatomy of the subject 150, and may be in the form of an implanted device, a swallow-able sensor (e.g., a pill), or the like.

Medical imaging data from an ultrasound transducer, e.g., ultrasound data, generally includes data associated with the internal structure of a portion of the subject's anatomy that was generated via the application of ultrasound to the anatomy of the subject, whereby pulses of high frequency vibration are transmitted into tissue using a probe, e.g., an ultrasound transducer. The vibrations reflect, at least partially, from surfaces that represent a change in acoustic impedance within the body, e.g., a geometry of a structure or tissue. Reflected vibrations that return to the transducer may be transmitted, e.g., to the data storage system 130. The generation of image data and/or a medical imaging reconstruction using ultrasound data is generally based on the time taken by the reflections to return to the transducer after application of the vibrations, and the intensity of the returned reflections. A conventional transducer generally is configured to receive variance in signal response across only one dimension. In other words, for a static location of a transducer, only one column of pixel data for an ultrasound image may be received. Thus, in order to generate an image or model of anatomy, a single transducer is generally swept over a field of view, e.g., rotated back and forth, in order to successively add and/or refresh columns of values to the data.

As noted above, in some embodiments, the first medical imaging device 120 may include a transducer array. A transducer array may include, for example, a plurality of transducers arranged in parallel with each other, e.g., distributed over at least a portion of an outer circumference of the distal end of medical device 115 and/or first medical imaging device 120, or in an approximately linear shape along the distal end medical device 115 and/or first medical imaging device 120. As a result, multiple columns of data may be sensed at once without rotation. In various embodiments, any suitable number and/or type of transducers in parallel may be used. Additional transducers effectively increase a static field of view.

Although some of the examples above pertain to ultrasound, it should be understood that any suitable modality or modalities of medical imaging may be used by or with the first medical imaging device 120 such as, for example, an optical sensor, an optical coherence tomography device, an infra-red sensor, a spectrometer, etc.

FIG. 2 depicts an exemplary embodiment of a first medical imaging device integrated into a medical device 115. However, it should be understood that the embodiment in FIG. 2 is illustrative only, and that any suitable medical device may be used, e.g., a medical device that does not include a medical imaging device. The medical device 115 in FIG. 2 may include a distal end 205 connected to a proximal end 210 via a shaft 215.

The distal end 205 may include one or more portions 220 configured to one or more of receive a component or communicate with a lumen (e.g., a working channel) disposed in the shaft 215. For example, the first medical imaging device 120 may be disposed in one of the portions 220. In another example, a tool having an end effector 230 may be disposed in another of the portions 220, e.g., an ablation device, a needle, a knife, forceps, a snare, a balloon, a delivery device, a guidewire, a net, an orifice for taking in or outputting fluid and/or material, etc. Distal end 205 also may include, for example, one or more lights, image sensors, openings for air/water/suction, an elevator, or any other suitable features.

The shaft 215 may, in some embodiments, include a flexible material. The shaft 215 may include one or more lumens (not shown) that communicate between the distal end 205 and the proximal end 210. In some embodiments, the shaft 215 may further include and/or house other elements such as a wire connector configured to communicate data between a component at the distal end 205, e.g., a position sensor 225, imaging device 120, a camera, or a light, and the proximal end 210.

The proximal end 210 may include, for example, a handle portion 245 that enables an operator to manipulate, advance, retract, and/or orient the distal end 205. For example, handle portion 245 may include one or more actuators (e.g., knobs) 255 that are used to articulate/steer the distal end 205, as well as actuators to deliver fluids/suction, or perform other controls. The proximal end 210 may further include one or more interfaces 250, e.g., an umbilicus to output data, send or receive electrical signals, and/or communicate a fluid or material into or out from the medical device 115. An interface for data may include one or more of a wired or wireless connections. The interface 250 may also be configured to receive power for operating the first medical imaging device 120, the position sensor 225, the end effector 230, a camera, a light, or another feature of distal end 205. In some embodiments, the medical device 115, or at least a portion thereof, is configured to be disposable, e.g., a single-use device.

In another exemplary embodiment (not shown), a proximal end of the medical device 115 may include or be configured to operate with an actuator (e.g., an actuator 127 from FIG. 1) that is operable, e.g., via electronic control, to manipulate the distal end 205. For example, the proximal end of medical device 115 may include features such that the proximal end may be received by the actuator 127, or may include an actuator 127 integrated therein. In other words, in some embodiments, the distal end 205 may be remotely and/or robotically controlled. Medical device 115 may include any features suitable for remote and/or robotic control.

Returning to FIG. 1, the second medical imaging device 125 may be configured to capture second medical imaging data from outside of the subject. In various embodiments, the second medical imaging device 125 may include, for example, a percutaneous ultrasound device, an X-Ray scanner, a CT scanner, or the like. In some embodiments, the second medical imaging device 125 may be at least partially electronically controlled. In some embodiments, a sensor of the second medical imaging device 125 may be movable and/or orientable, e.g., via the operator 145 or via electronic control. For example, a sensor of the second medical imaging device 125 may be mounted on a movable arm (not shown). The movable arm may, for example, be manually manipulated, and/or may include one or more of the actuators 127 that enable electronic control of the positon and/or orientation of the sensor of the second medical imaging device 125, e.g., to target different regions of the anatomy of the subject 150. In some embodiments, the second medical imaging device 125 may include a position sensor 165 that, similar to the position sensor 160 for the first medical imaging device 120, may be usable to determine a position or orientation of the second medical imaging device 125 and/or a region of the anatomy of the subject targeted by the second medical imaging device 125.

In some embodiments, the second medical imaging device 125 may have a settable field of view that enables a user 140 to select one or more of a targeted region or orientation. Any suitable external medical imaging device may be used. For example, in some embodiments, the second medical imaging device 125 may include a plurality of sensors or arrays of sensors.

FIG. 3 depicts an exemplary schematic for a second medical imaging device 125 that includes a plurality of sensor arrays 300. Each array 305 in the plurality of arrays 300 includes a plurality of sensors 310, e.g., ultrasound transducers. In an exemplary embodiment, the plurality of sensor arrays 300 may be disposed over a subject 150. By activating and/or deactivating different arrays 305 of the plurality of arrays 300, different portions of the anatomy of the subject 150 may be imaged, e.g., without having to move or reorient the second medical imaging device. In the embodiment illustrated in FIG. 3, the plurality of arrays 300 are arranged in a cross shape. However, any suitable arrangement may be used such as, for example, any polygonal or irregular shape. In some embodiments, the plurality of arrays 300 may be shaped in a manner corresponding to at least a portion of human anatomy, e.g., shaped to correspond to a human torso shape or the like. Additionally, the arrays 305 may have any suitable shape (rectangle, triangle, circle, irregular, etc.) Although depicted as separate in FIG. 3, in some embodiments, one or more arrays 305 may at least partially overlap. Further, in some embodiments, individual sensors 310 may be separately addressable, e.g., so that division of sensors into different arrays 305 is controlled electronically. In some embodiments, different arrays 305 may be tilted, e.g., toward each other or away from each other or in irregular directions. Such tilting may reduce cross talk between sensor signals of different arrays, e.g., when multiple arrays 305 are used at once, and/or may enable a different orientation for imaging of the anatomy of the subject 150. Although some of the embodiments above were described in which the plurality of arrays 300 remains in a static position relative to the subject 150, in some embodiments the plurality of arrays 300 may be movable, e.g., either manually or via an electronically controlled actuator, e.g., the actuator 127 in FIG. 1. For example, the plurality of arrays 300 may be translatable, rotatable, and/or movable in a vibrating or oscillating motion.

Returning to FIG. 1, the data storage system 130 may include or be accessible via a medical data exchange system, or the like. The data storage system 130 may include a server system, an electronic medical data system, computer-readable memory such as a hard drive, flash drive, disk, etc. In some embodiments, the data storage system 130 includes and/or interacts with an application programming interface for exchanging data to other systems, e.g., one or more of the other components of the environment. The data storage system 130 may include and/or act as a repository or source for medical imaging data. For example, medical imaging data resulting from a medical imaging device may be stored by the data storage system 130 and/or provided by the data storage system 130 to the reconstruction system 155 as discussed in more detail below.

In various embodiments, the electronic network 135 may be a wired connection between devices, a wireless connection between devices, a wide area network (“WAN”), a local area network (“LAN”), personal area network (“PAN”), or the like. In some embodiments, electronic network 135 includes the Internet, and information and data provided between various systems occurs online. “Online” may mean connecting to or accessing source data or information from a location remote from other devices or networks coupled to the Internet. Alternatively, “online” may refer to connecting or accessing an electronic network (wired or wireless) via a mobile communications network or device. The Internet is a worldwide system of computer networks—a network of networks in which a party at one computer or other device connected to the network can obtain information from any other computer and communicate with parties of other computers or devices. The most widely used part of the Internet is the World Wide Web (often-abbreviated “WWW” or called “the Web”). A “website page” generally encompasses a location, data store, or the like that is, for example, hosted and/or operated by a computer system so as to be accessible online, and that may include data configured to cause a program such as a web browser to perform operations such as send, receive, or process data, generate a visual display and/or an interactive interface, or the like.

As discussed in further detail below, the reconstruction system 155 may perform operations relating to generating and/or updating medical imaging reconstructions based on medical imaging data, monitoring medical imaging data captured by medical imaging devices, e.g., to determine a divergence, guiding and/or operating medical imaging devices and/or medical devices, as well as other activities. The reconstruction system 155 may include, for example, a server system, a desktop computer, a laptop, a tablet, etc. In an exemplary embodiment, the reconstruction system 155 includes a cloud-based architecture, e.g., for performing operations that include data processing, and may further include a display and/or an interface, e.g., that are accessible to the operator 145 during a medical procedure.

The reconstruction system 155 may include software and/or hardware that facilitates operations such as those discussed above. For example, the reconstruction system 155 may include one or more algorithms, models, or the like for generating medical imaging reconstructions based on medical imaging data. In various embodiments, the reconstruction system 155 may facilitate generating medical imaging reconstructions in one or more of a handcrafted (e.g., user-driven) manner, based on one or more baseline models, using a machine-learning driven model, e.g., a deep-learning model, or the like. Any suitable technique or combination of techniques for generating medical imaging reconstructions may be used.

In an exemplary embodiment, the reconstruction system 155 may include a first reconstruction algorithm, model, or the like that is configured to access medical imaging data captured by the pre-procedure medical imaging device 110, e.g., from the data storage system 130, and generate an initial medical imaging reconstruction based on the pre-procedure medical imaging data. In a further exemplary embodiment, the reconstruction system 155 may include one or more registration algorithms configured to determine a registration between different instances of medical imaging data. For example, medical imaging data received from the first or second medical imaging devices 120 and 125 may be registered with each other and/or with an existing medical imaging reconstruction, such as the initial medical imaging reconstruction discussed above.

The reconstruction system 155 may further include one or more monitoring algorithms configured to monitor medical imaging data received from the first or second medical imaging devices 120 and 125. For example, one or more algorithms may be used to determine one or more characteristics of obtained medical imaging data such as, for example, what region of the anatomy of the subject 150 is targeted, a relative location of the targeted region to other regions targeted by other medical imaging, an age of the medical imaging data, a position of the medical imaging device used to capture the obtaining medical imaging data, a relative positon of the medical imaging device relative to other medical imaging devices, a position of a medical device, etc. The reconstruction system 155 may include one or more algorithms for determining a divergence in medical data, e.g., based on the monitoring.

The reconstruction system 155 may include one or more algorithms for guiding or operating medical imaging devices, e.g., by identifying one or more regions of anatomy to be imaged, operating an actuator 127 or the like to reposition or reorient a medical imaging device, etc. In an example, the reconstruction system 155 may be configured to cause a medical imaging device to move according to a predetermined route or pattern. The reconstruction system 155 may include one or more algorithms for updating and/or outputting information related to a medical imaging reconstruction, e.g., that accounts for data recency of the medical imaging data received by the reconstruction system 155. Further aspects of the foregoing are discussed in further detail below.

Although depicted as separate components in FIG. 1, it should be understood that a component or portion of a component in the environment may, in some embodiments, be integrated with or incorporated into one or more other components. For example, a portion of the reconstruction system 155 may be integrated into the user device 105 or the data storage system 130. In some embodiments, operations or aspects of one or more of the components discussed above may be distributed amongst one or more other components. Any suitable arrangement and/or integration of the various systems and devices of the environment may be used.

Further aspects of the registration system 155 and/or how it may be utilized in conjunction with a medical device 115 and/or a medical procedure to provide information regarding data recency of captured medical imaging data are discussed in further detail in the methods below. In the following methods, various acts may be described as performed or executed by a component from FIG. 1, such as the user device 105, the first or second medical imaging devices 120 and 125, the registration system 155, or components thereof. However, it should be understood that, in various embodiments, various components of the environment discussed above may execute instructions or perform acts including the acts discussed below. An act performed by a device may be considered to be performed by a processor, actuator, or the like associated with that device. Additionally, in various embodiments, various operations may include or enable user control, e.g., via the operator 145. Further, it should be understood that in various embodiments, various steps may be added, omitted, and/or rearranged in any suitable manner.

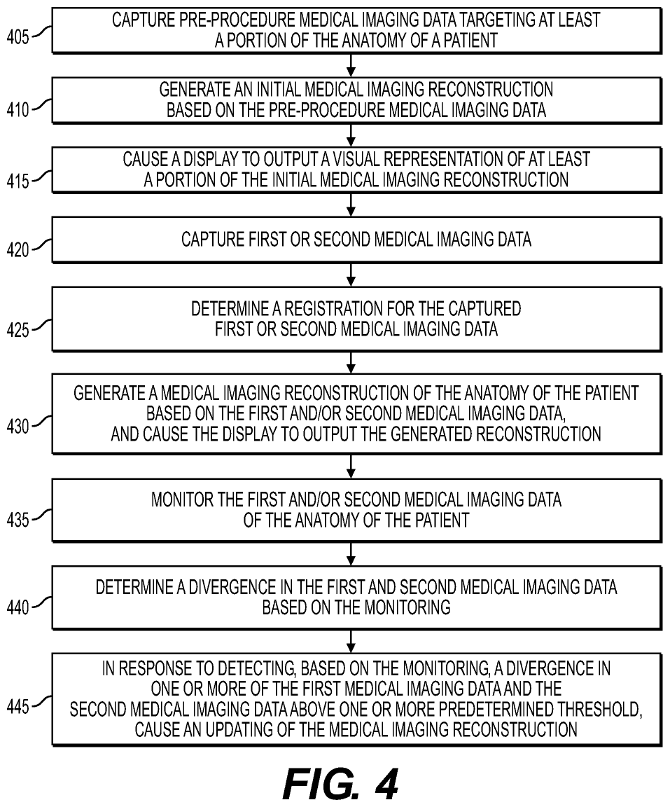

FIG. 4 illustrates an exemplary process for providing a medical imaging reconstruction of a subject's anatomy that accounts for data recency of medical imaging data used for the reconstruction. At step 405, a pre-procedure medical imaging device 110 may be used to capture pre-procedure medical imaging data targeting at least a portion of the anatomy of a subject 150. For example, an operator 145 may use an MRI scanner to target the portion of the anatomy of the subject 150 and capture MRI data. The pre-procedure medical imaging data may be stored on the data storage system 130, e.g., that is accessible to the pre-procedure medical imaging device 110 via an electronic network 135. At step 410, a reconstruction system 155 may generate an initial medical imaging reconstruction based on the pre-procedure medical imaging data. Any suitable medical imaging reconstruction technique may be used.

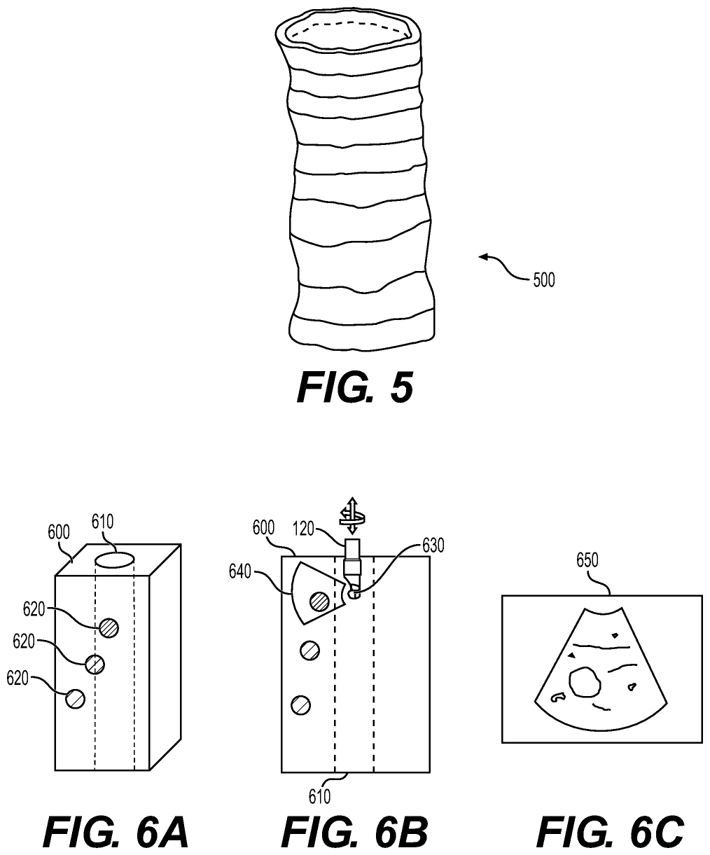

At step 415, the reconstruction system 155 may cause a display to output a visual representation of at least a portion of the initial medical imaging reconstruction. For example, the display may be a display accessible to the operator 145 performing a medical procedure on the subject 150. FIG. 5 illustrates an exemplary embodiment of a visual representation 500 of a segmented CT reconstruction, e.g., a medical imaging reconstruction generated based on segments or slices of imaging of the subject's anatomy taken via a CT scanner.

Returning to FIG. 4, at step 420, one or more of a first medical imaging device 120 or a second medical imaging device 125 may capture first or second medical imaging data, respectively, e.g., during the course of the procedure. Medical imaging data may be captured continuously, intermittently, on request or action of an operator 145, automatically (e.g., via electronic control of the reconstruction system 155 or the like), etc. The captured medical imaging data may be stored on the data storage system 130.

In some embodiments, one or more of the operator 145 or the reconstruction system 155 may reposition or reorient one or more of the first or second medical imaging devices 120 or 125, e.g., via manual manipulation, re-targeting focus, or electronic control via one or more actuator 127. For example, if the procedure being performed pertains to a particular region of the anatomy of the subject 150, one or more of the first or second medical imaging devices may be repositioned and/or reoriented to target the particular region.

At step 425, the reconstruction system 155 may determine a registration for the captured medical imaging data, e.g., a spatial and/or geometric mapping, relationship, or translation between data. In some embodiments, the captured medical imaging data may be registered relative to the initial reconstruction. In some embodiments, e.g., in embodiments without pre-procedure medical imaging, the captured medical imaging data may be registered relative to a baseline model, may be registered manually, e.g., by the operator 145, etc. In some embodiments, the first medical imaging data may be registered relative to the second medical imaging data. A registration may be used to determine one or more characteristics of the captured medical imaging data such as, for example, one or more regions of the anatomy of the subject targeted by the medical imaging device(s), a position of the medical imaging device(s) relative to each other and/or to the anatomy of the subject, etc. In some embodiments, the registration system 155 may obtain or record additional data along with captured medical image data. Such additional data may include, for example, metadata describing one or more of a time at which the data was captured, one or more characteristics of the medical imaging device(s) used to capture the data (e.g., position or targeting data based on feedback from an actuator 127), one or more characteristics of the anatomy of the subject (e.g., determined based on the captured data via an analysis algorithm) or the like.

In an example, the anatomy of a subject 150 may include one or more structures of interest, e.g., a lesion, a wound, a foreign body, diseased tissue, or the like. FIG. 6A depicts a schematic representation of a portion of the anatomy 600 of the subject 150, which includes a lumen 610 by which the first medical imaging device 120 may be navigated, and several lesions 620 to be investigated during the procedure.

As depicted in FIG. 6B, an exemplary embodiment of a first medical imaging device 120 that is separate from a medical device 115 may be navigated through the lumen 610. It should be understood that in various embodiments, any suitable first medical imaging device may be used. In some embodiments, first medical imaging data may be captured as the first medical imaging device 120 is navigated. In some embodiments, first medical imaging data may be captured continuously, periodically, upon request, or the like. In this example, the first medical imaging device 120 may include an ultrasound transducer 630 configured to image a portion of the tissue 640 bordering the lumen 610. FIG. 6C depicts an exemplary embodiment of an “ultrasound fan,” e.g., a visual depiction 650 of ultrasound data that may be captured via the ultrasound transducer 630, e.g., by sweeping the ultrasound transducer 630 over an angle of rotation when the first medical imaging device 120 is positioned at an area of interest and/or activating a parallel array of transducers, or the like.

In some embodiments, the captured first medical imaging data may be analyzed, e.g., to identify a lesion, a wound, a foreign body, diseased tissue, a location of a medical device 115, or the like present therein. For example, in some embodiments, the reconstruction system 155, a separate classification system, the operator 145, or the like may perform one or more analysis on the first medical imaging data. Any suitable analysis technique may be used, e.g., analysis by the operator 145, analysis using an automated technique such as machine learning, or the like. Any identified structures of interest may be associated with the registration, e.g., to identify a location of the identified structure in the reconstruction.

The visual depiction in FIG. 6C represents a single instance of capturing first medical imaging data using the first medical imaging device 120, e.g., a single instance of sweeping the ultrasound transducer 630 over an area of interest, a single instance of operating a phased array of transducers, or the like. Also, while the first medical imaging device 120 was discussed with regard to FIGS. 6A-C above, it should be understood that the second medical imaging device may capture second medical imaging data of the anatomy of the subject 150 instead of and/or in addition to first medical imaging data captured by the first medical imaging device 120. In various embodiments, the first and/or second medical imaging devices 120 and 125 may capture multiple instances of medical imaging data. For example, first medical imaging data of first medical imaging device 120 may be captured at various locations as the first medical imaging device 120 is navigated through the lumen 610. In various embodiments, medical imaging data may be captured continuously, in discrete samples, or any combination thereof.

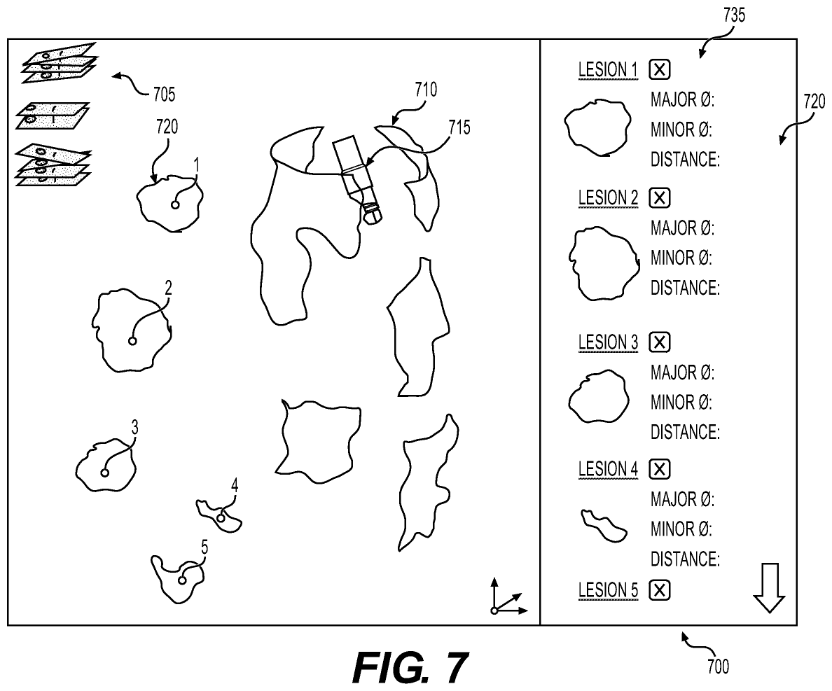

Returning again to FIG. 4, at step 430, the reconstruction system 155 may generate a medical imaging reconstruction of the anatomy of the subject based on the first and/or second medical imaging data, and may cause the display to output the generated reconstruction. In an example, the reconstruction system 155 may generate a new medical imaging reconstruction based on the first and/or second medical imaging data. FIG. 7 depicts an exemplary embodiment of a graphical user interface 700 that may be output by the reconstruction system 155. The interface 700 illustrates that multiple instances of data 705 have been captured, and have been used to generate a medical imaging reconstruction 710 based thereon. As shown in FIG. 7, the instances 705 may be illustrated with orientations showing how they relate to each other in three-dimensional space.

The interface 700 may also illustrate a current location 715 of the first medical imaging device 120 within the reconstruction 710, e.g., based on the determined registration, a position sensor 160, tracking based on a second medical imaging device 125, or the like. The interface 700 may also depict structures of interest 720 identified, for example, by an analysis algorithm, such as lesions 1-5. In various embodiments, the interface 700 may further include other data or views of the reconstruction 710. For example, in an embodiment, the interface 700 may include an axial view of the lumen 610 (e.g., a cross-section view) along with a direction indicator indicating a current orientation of the first medical imaging device 120 (e.g., which direction a sensor of the first medical imaging device 120 is facing in the plane of the cross-section view).

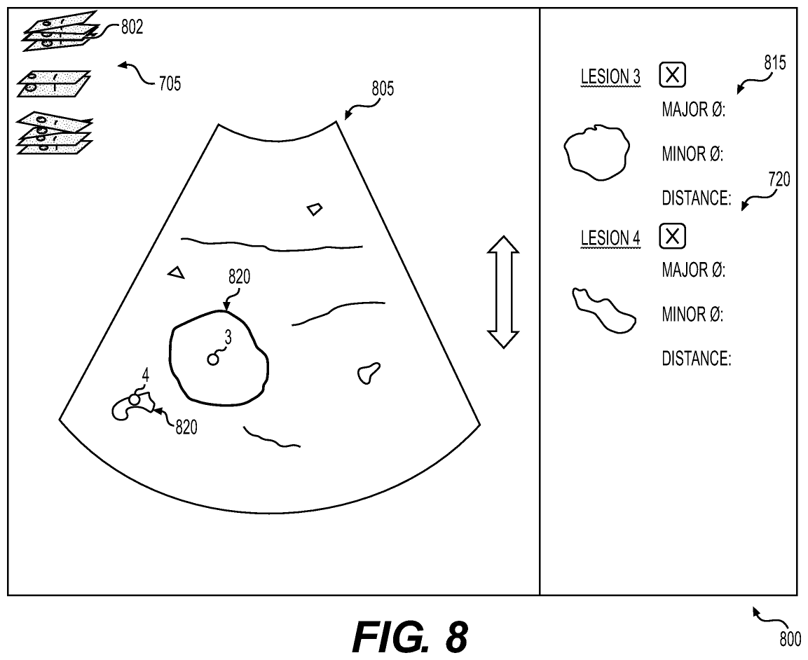

In some embodiments, the interface 700 may include user-selectable options, such as interface items 735 for displaying or hiding selected structures of interest 720. In some embodiments, one or more of the instances 705, or the structures of interest 720 may be user selectable. FIG. 8 depicts an exemplary embodiment of a display 800 for the interface 700 after a user 140 selects a particular instance 802 from amongst the instances 705 of medical imaging data. The display 800 may include a visual representation 805 of the selected instance 705 of medical imaging data (e.g., an ultrasound fan when the instance is ultrasound data), a display 815 of which structures of interest 720 are visible in the particular instance 705, and a visual highlighting 820 of the visible structures of interest 720 visible in the visual representation 805. In some embodiments, the display 800 may enable a user 140 to select portions of the visual representation, e.g., to add, remove, or edit a structure of interest 720.

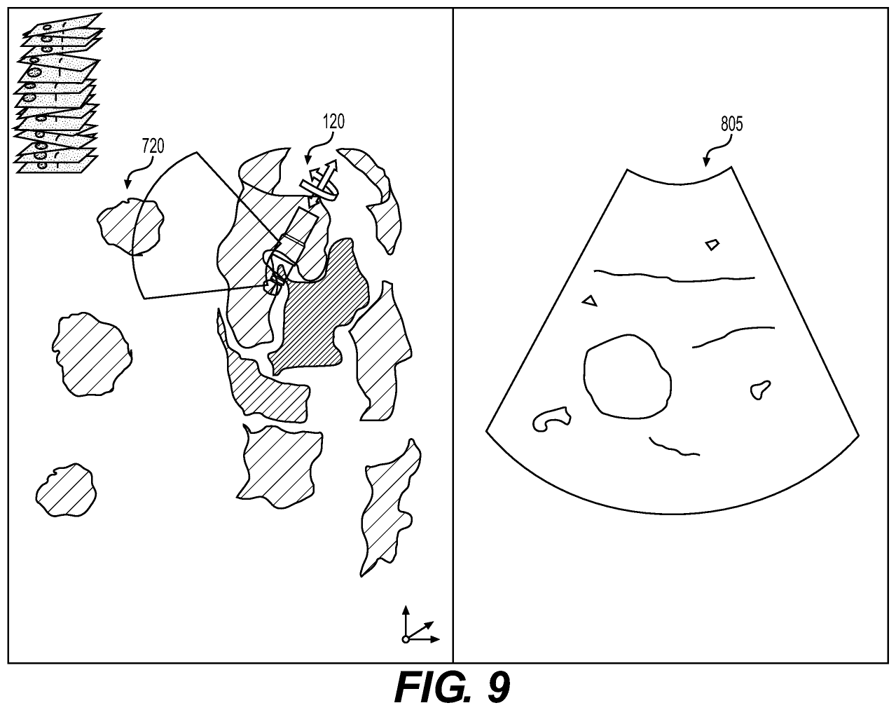

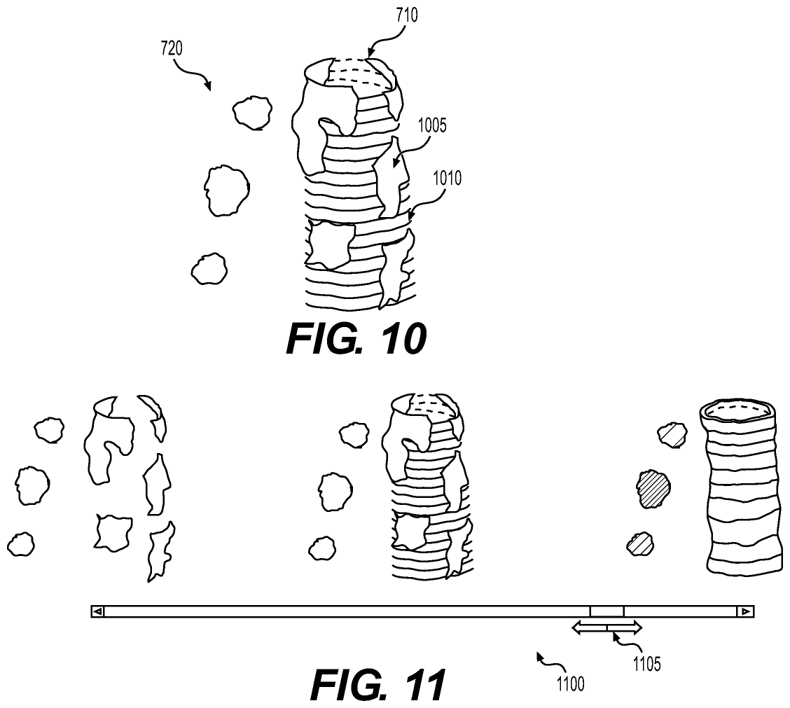

As further medical imaging data is captured, the reconstruction system 155 may update the interface 700 with additional instances 705, and with updates to the reconstruction 710. FIG. 9 illustrates an exemplary embodiment of the interface 700 in which the first medical imaging device 120 have been navigated back to a previously imaged region, e.g., in order to capture additional information regarding structure of interest 720 so that the medical imaging reconstruction 710 includes additional information near the structure of interest 720. In the interface 700 illustrated in FIG. 9, a visual depiction 805 of a current instance of first medical imaging data is also shown.

In some embodiments, e.g., embodiments in which pre-procedure medical imaging data is available, the reconstruction system 155 may include information from the pre-procedure medical imaging data in the reconstruction 710. The pre-procedure imaging data may be used, for example, to generate a reconstruction having a wider field of view or a more inclusive imaging of a region of anatomy relevant to the procedure. Such a reconstruction may act as a general mapping guide, e.g., to provide the operator 145 with a general understanding of the anatomy relevant to the procedure supplemented with imaging data form the first and second medical imaging device 120 and 125. FIG. 10 depicts an exemplary embodiment of a visual depiction of a reconstruction 710 that includes a first portion 1005 based on the first and/or second medical imaging data and a second portion 1010 based on the pre-procedure medical imaging data.

In some embodiments, the reconstruction system 155 may be configured to vary a relative visibility of the first and/or second medical imaging data to the pre-procedure medical imaging data. FIG. 11 depicts an exemplary embodiment of an interface 1100 that includes a user-settable slider 1105 that is operable to select a relative visibility of the first and/or second medical imaging data to the pre-procedure medical imaging data. For example, moving the slider 1105 leftwards increases the visibility of the first and/or second medical imaging data and/or decreases the visibility of the pre-procedure medical imaging data, such that one or the other or both may be displayed. In another example, moving the slider 1105 rightwards decreases the visibility of the first and/or second medical imaging data and/or increases the visibility of the pre-procedure medical imaging data, such that one or the other or both may be displayed.

Turning back to FIG. 4, at step 435, the reconstruction system 155 may monitor the first and/or second medical imaging data of the anatomy of the subject 150. In one example, the reconstruction system 155 may monitor a position of the first and/or second medical imaging devices 120 or 125, may monitor a location or locations targeted by the first and/or second medical imaging devices 120 or 125, and/or may monitor an extent of time elapsed since different instances of the first and/or second medical imaging data were captured. In some embodiments, the reconstruction system may include additional data such as a status or feedback signal from one or more actuator 127 and/or position sensor 160 or 165, a location of the medical device 115, or the like in the monitoring.

At step 440, the reconstruction system 155 may determine a divergence in the first and second medical imaging data based on the monitoring. Divergence may be based on, e.g., associated with, one or more factors, such as the examples below.

In some embodiments, the divergence may be determined based on a distance and/or a change in distance between a first region of the anatomy of the subject 150 targeted by the first medical imaging device 120 and a second region of the anatomy of the subject 150 targeted by the second medical imaging device 125. For example, in an exemplary use case, the second medical imaging device 125 may be used to perform outside imaging of a region of the anatomy of the subject 150 believed to be relevant to a procedure. During the course of the procedure, an operator 145 may navigate the first medical imaging device 120 and/or the medical device 115 toward a portion of the anatomy outside of that region. Thus, the distance and/or change in distance between regions targeted by the first and second medical imaging devices 120 and 125 may be indicative of a divergence in the first and second medical imaging data.

In some embodiments, the divergence may be determined based on a distance or change in distance between a first location of the first medical imaging device 120 and a second location of the second medical imaging device 125. For example, in an exemplary use case, it may be desirable to have the regions targeted by the first and second medical imaging devices 120 and 125 to be proximate to each other and/or to overlap. Thus, the distance and/or change in distance between locations of the first and second medical imaging devices 120 and 125 may be indicative of a divergence in the first and second medical imaging data.

In some embodiments, the divergence may be determined based on a distance or change in distance between a location of a portion of the medical imaging reconstruction 710 (e.g., a portion having high confidence of validity as discussed further below) and a location of a medical device 115. For example, the operator 145 may navigate the medical device 115 to a region with less confidence of validity in the reconstruction 710 and/or out of bounds of the reconstruction 710.

In some embodiments, the divergence may be based on a moving average of the distance between locations of the first and second medical imaging devices 120 and 125, a moving average of the distance between regions targeted by the first and second medical imaging devices 120 and 125, and/or a moving average of the distance between the medical device 115 and the portion of the medical imaging reconstruction 710. The use of an average term such as a moving average may cause the divergence to be less susceptible to noise and/or rapid or aberrant movements by one of the first and second medical imaging devices 120 and 125. Any other suitable smoothing or noise attenuating technique may also be used when determining a distance such as in the examples above. As will be discussed in further detail below, such smoothing may reduce jitter in the operation of the medical imaging devices, facilitate subject safety, and reduce a risk of imaging unwanted or unnecessary portions of the anatomy of the subject 150.

In some embodiments, when determining a distance, such as in one or more of the foregoing examples, the reconstruction system 155 may use a signal received from one or more position sensor 160 or 165. For example, the first medical imaging device 120 may include a position sensor 160 in the form of an electromagnetic position sensor. In other examples, such as in the embodiment discussed above with regard to FIG. 2, the first medical imaging device 120 may be integrated into a medical device 115 that includes a shaft 215 that includes a fiber optic channel, and the reconstruction system 155 may employ fiber optic shape sensing in order to location and/or orient the first medical imaging device 120.

In some embodiments, the reconstructions system 155 may determine a location of the first medical imaging device 120 using the second medical imaging data. For example, the reconstruction system 155 and/or another system may employ an analysis algorithm on the second medical imaging data to identify the first medical imaging device 120 in a region targeted by the second medical imaging device 125, and determine the location and/or orientation of the first medical imaging device 120 based on the identification and/or a registration of the second medical imaging device 125 with the anatomy of the subject 150. In some embodiments, feedback from an actuator 127 used to control the position or orientation of one or more of the first or second medical imaging devices 120 or 125 may be used. For example, feedback from an actuator 127 and/or an associated sensor may track how a medical imaging device is manipulated. In some embodiments, impedance-based position tracking may be used. For example, one or more electrodes may be placed at different locations on the body of the subject 150, an electrical current may be generated by an electrode included with the first medical imaging device 120, and the reconstruction system 155 may determine a location of the first medical imaging device 120 based on the different currents exhibited by the electrodes on the body of the subject 150. In some embodiments, a location of the second medical imaging device 125 may be determined based on a signal from a position sensor 165 in a manner similar to the examples provided above for the first medical imaging device 120. In some embodiments, the location of the second medical imaging device 125 may be determined based on optical tracking, e.g., via a signal from an optical sensor such as a camera disposed near the second medical imaging device 125.

In some embodiments, the divergence may be based on an extent of time elapsed since one or more instances of medical imaging data were captured. In other words, in some embodiments, the divergence may be based on an extent of time elapsed since one or more of the first medical imaging data was captured by the first medical imaging device or the second medical imaging data was captured by the second medical imaging device. As noted above, the state of the anatomy of the subject 150 may change over time for various reasons. The longer the period of time elapsed since a particular instance of medical imaging data was captured, the more likely that the particular instance may represent an at least partially inaccurate reflection of reality as to the anatomy of the subject. In other words, the validity and/or confidence for the particular instance of medical imaging data may decrease over time. Thus, the reconstruction system 155 may monitor how long has elapsed since each instance of medical imaging data was captured.

While various examples of the basis for divergence were discussed above, it should be understood that any suitable basis or combination thereof may be used. Further, in various embodiments, divergence may be determined respectively for each instance of medical imaging data, on a region by region basis of the anatomy (e.g., for a region that was imaged by a plurality of instances), separately for each medical imaging device, or any combination thereof. In some embodiments, divergence may be expressed in terms of a value (e.g., corresponding to a magnitude of a distance or of a period of time, etc.). In some embodiments, divergence may be based on a combination of such factors, and different quantities may be weighted or normalized via any suitable technique. For example, one point of divergence may accrue per second, in addition to one point of divergence for every inch of separation between the medical imaging devices. However, it should be understood that the foregoing example is illustrative only, and that any suitable scheme for using one or more of the factors discussed above to determine divergence may be used.

In some embodiments, the determined divergence may be compared to a predetermined threshold. Such comparison may occur, for example, in real time or near real time, periodically such as once per second, every 5 seconds, every minute, every two minutes, etc., or at any other suitable timing, in response to a request by the operator 145, in response to detection of an anatomical characteristic of the subject 150, such as detection of a structure of interest, in response to the reconstruction system 155 receiving a new instance of medical imaging data, or the like. Any suitable predetermined threshold may be used. In some embodiments, such as embodiments discussed in further detail below in which the display of the medical imaging reconstruction 710 to indicate the time elapsed relative to each instance of medical imaging data, the predetermined threshold may be any non-zero amount. In some embodiments, different divergences associated with different factors and/or different predetermined thresholds may be determined for different purposes. For example, a divergence based on elapsed time with a threshold of greater than zero may be determined for the purpose of updating the visual depiction of the medical imaging reconstruction, and a divergence based on elapsed time with a threshold greater than 5 minutes may be determined for causing a medical imaging device to reacquire medical imaging data, as discussed in further detail below.

At step 445, in response to detecting, based on the monitoring, a divergence in one or more of the first medical imaging data and the second medical imaging data above one or more predetermined threshold, the reconstruction system 155 may cause an updating of the medical imaging reconstruction 710.

In some embodiments, updating the medical imaging reconstruction 710 includes an updating of the medical imaging data used to form the medical imaging reconstruction. For example, in some embodiments, the reconstruction system 155 may cause at least one of the first medical imaging device and the second medical imaging device to reacquire first or second medical imaging data from one or more regions of the anatomy of the subject 150, and then modifying the medical imaging reconstruction 710 using the newly acquired data. In some embodiments, the first medical imaging device 120 may be operated by the operator 145, and thus the updating of the medical imaging reconstruction 710 may include causing a reacquisition of medical imaging data by the second medical imaging device 125, which may be autonomously controlled electronically by the reconstructions system 155. For example, the reconstruction system 155 may operate one or more actuators 127 to reposition and/or reorient one of the medical imaging devices. In some embodiments, the reconstruction system 155 may output a message or visual prompt, e.g., to the operator 145 and via a display, that includes an instruction for operating one of the medical imaging devices to reacquire medical imaging data. In various embodiments, the first medical imaging device 120 may be electronically controlled, or both or neither of the first and second medical imaging devices 120 and 125 may be electronically controlled.

In some embodiments, the one or more regions to be imaged and/or re-imaged for the updating may include a region that would decrease the divergence. For example, a region corresponding to an instance of medical imaging data that was captured some time ago may be reimaged, so as to decrease a time elapsed since that region was imaged. In another example, the second medical imaging device 125 may be moved or reoriented so as to image a region closer to a location of the first medical imaging device 120 or closer to a region targeted by the medical imaging device, or vice versa.

In some embodiments, the one or more regions may include a plurality of regions, and the reacquisition of medical imaging data may include targeting the plurality of regions according to a sequence and/or pattern. For instance, in an exemplary embodiment, the second medical imaging device 125 may include a transabdominal, transthoracic, or conventional ultrasound sensor or the like and a robotic arm controlled by one or more actuator 127. The reconstruction system 155 may cause the second medical imaging device 125 to reacquire second medical imaging data for a plurality of regions, e.g., the regions relevant to the procedure, and may operate the one or more actuators 127 to manipulate the second medical imaging device 125 according to one or more patterns that cause the second medical imaging device 125 to successively target each of the plurality of regions.

Any suitable pattern may be used such as, for example, a random or pseudo-random pattern, a linear pattern, a spiral pattern, a movement scheme predetermined by a decision algorithm, or the like. In some embodiments, a pattern may include one or more of tilting, rotating, sweeping, or pivoting the second medical imaging device 125. In some embodiments, the reconstruction system 155 may, instead of or in addition to the applying of a pattern such as the foregoing, apply an obstacle avoidance protocol to a medical imaging device 125. For example, one or more of the actuators 127 may include or be associated with one or more force transducer. In response to a signal from a force transducer indicative of a force over a predetermined threshold, the reconstruction system 155 may, one or more of, halt manipulation of the medical imaging device, identify an obstacle and cause the medical imaging device to be manipulated around the obstacle, modify the pattern, or the like.

In another exemplary embodiment, as discussed above, the second medical imaging device 125 may include a plurality of sensors or arrays of sensors 300 (FIG. 3), and causing the second medical imaging device 125 to reacquire medical imaging data from one or more regions of the anatomy of the subject 150 may include one or more of changing which sensors or arrays 305 of the plurality of sensors or arrays 300 are activated, changing a position or orientation of the plurality of sensors or arrays 300, or the like, so as to target one or more regions of the anatomy of the subject 150.

In some embodiments, updating the medical imaging reconstruction 710 includes modifying a visual depiction of the medical imaging reconstruction 710. For example, in some embodiments, the updating may include modifying the medical imaging reconstructions so as to include and/or account for newly acquired medical imaging data, and then updating the visual depiction of the medical imaging reconstruction accordingly 710. In some embodiments, the updating may include modifying the visual depiction of at least a portion of the medical imaging reconstruction such that at least a portion of the medical imaging reconstruction has a visual appearance indicative of an extent of time elapsed since medical imaging data upon which the at least portion is based was captured. For example, the first and second medical imaging devices 120 and 125 may capture first and second medical imaging data from various regions at various times. The visual depiction of the medical imaging reconstruction 710 may, as time passes, gradually change the visual appearance of each region depending on how much time has elapsed since that particular region was imaged.

A visual appearance indicative of an extent of time elapsed may include, for example, a reduction in opacity, contrast, saturation and/or brightness, in which a magnitude of the reduction increases with an increase in the extent of time elapsed. For instance, in FIG. 9, regions of the reconstruction which are more recent are depicted with one or more of a different color or saturation, relative to less recently imaged regions. In another example, the visual appearance may include one or more of a reduction in sharpness or resolution, in which a magnitude of the reduction increases with an increase in the extent of time elapsed. In an illustrative embodiment, the magnitude to which the resolution or sharpness is decreased may be configured to correlate to an amount to which confidence in validity of the imaging for that region decreases, e.g., so that a high level of confidence may be maintained for the depiction of the region. In another example, the visual appearance may include a shift in color, in which a magnitude of the shift increases with an increase in the extent of time elapsed. In another example, the visual appearance may include a flashing effect, which may initiate when an extent of time since capture reaches a predetermined threshold, and/or which may accelerate/decelerate over time. In another example, the visual appearance may include a bounding region or selection, e.g., to visually indicate that the region or regions have a lower confidence or validity.

In another example, the visual appearance may include hiding regions for which a period of time since being imaged over a predetermined threshold has elapsed. In other words, the reconstruction system 155 may restrict the medical imaging reconstruction 710 to be based only on data captured within the predetermined threshold amount of time. In some embodiments, such hiding may only be applied to data captured by the first and second medical imaging device 120 and 125, e.g., so that pre-procedure medical imaging is still visible.

In some embodiments, the reconstructions system 155 may determine, e.g., based on the determined divergence, that the medical device 115 is located in a region for which the divergence is above the predetermined threshold, e.g., a region for which the confidence of validity is low. In some embodiments, the reconstruction system 155 may output a notification to the operator 145, e.g., a visual or auditory message, e.g., via the display, or a tactile response, e.g., via an actuator connected to the medical device 115. In some embodiments, the reconstruction system 155 may, one or more of, output instructions or prompts for the operator 145 to guide the medical device 115 to a region with higher confidence of validity, output a further notification when the medical device 115 is in a region with higher confidence of validity, cause one or more of the first or second medical imaging devices 120 or 125 to target a region closer to the medical device 115, or the like.

It should be understood that embodiments in this disclosure are exemplary only, and that other embodiments may include various combinations of features from other embodiments, as well as additional or fewer features. For example, while some of the embodiments above pertain to ablation of tissue within the periphery of the lung, any suitable procedure may be used. Further, while some of the embodiments above pertain to ultrasound, any suitable image modality or technique may be used.

In general, any process or operation discussed in this disclosure that is understood to be computer-implementable, such as the processes illustrated in FIG. 4 may be performed by one or more processors of a computer system, such any of the systems or devices in the environment of FIG. 1, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

A computer system, such as a system or device implementing a process or operation in the examples above, may include one or more computing devices, such as one or more of the systems or devices in FIG. 1. One or more processors of a computer system may be included in a single computing device or distributed among a plurality of computing devices. A memory of the computer system may include the respective memory of each computing device of the plurality of computing devices.

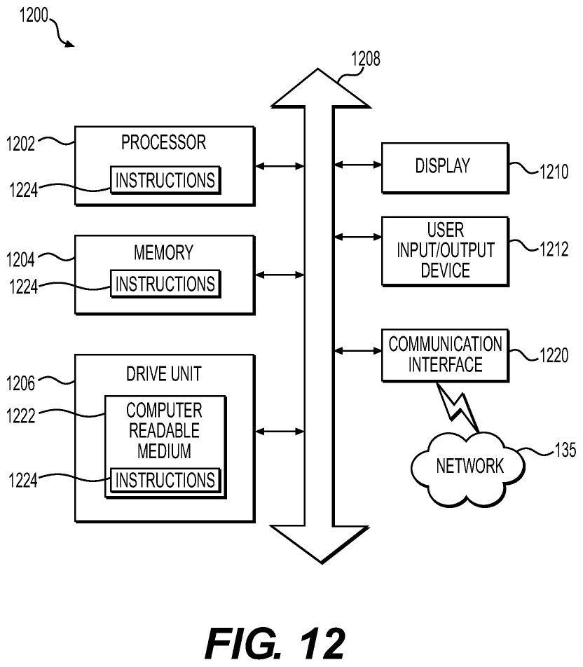

FIG. 12 is a simplified functional block diagram of a computer 1200 that may be configured as a device for executing the method of FIG. 4, according to exemplary embodiments of the present disclosure. For example, the computer 1200 may be configured as the reconstruction system 155 and/or another system according to exemplary embodiments of this disclosure. In various embodiments, any of the systems herein may be a computer 1200 including, for example, a data communication interface 1220 for packet data communication. The computer 1200 also may include a processor 1202, in the form of one or more processors or central processing unit (“CPU”), for executing program instructions. The computer 1200 may include an internal communication bus 1208, and a storage unit 1206 (such as ROM, HDD, SDD, etc.) that may store data on a computer readable medium 1222, although the computer 1200 may receive programming and data via network communications. The computer 1200 may also have a memory 1204 (such as RAM) storing instructions 1224 for executing techniques presented herein, although the instructions 1224 may be stored temporarily or permanently within other modules of computer 1200 (e.g., processor 1202 and/or computer readable medium 1222). The computer 1200 also may include input and output ports 1212 and/or a display 1210 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may, at times, be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While the disclosed methods, devices, and systems are described with exemplary reference to transmitting data, it should be appreciated that the disclosed embodiments may be applicable to any environment, such as a desktop or laptop computer, an automobile entertainment system, a home entertainment system, etc. Also, the disclosed embodiments may be applicable to any type of Internet protocol.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

What is claimed is:

1. A computer-implemented method for medical imaging reconstruction of a subject's anatomy, comprising:
monitoring first medical imaging data of the subject's anatomy and second medical imaging data of the subject's anatomy, wherein:
the first medical imaging data is captured from within the subject's anatomy by at least one first medical imaging device;
the second medical imaging data is captured from outside of the subject's anatomy by at least one second medical imaging device; and
at least one of the first medical imaging device or the second medical imaging device is operable to selectively target different regions of the subject's anatomy; and
in response to detecting, based on the monitoring, a divergence in the first medical imaging data and the second medical imaging data above a predetermined threshold, updating a medical imaging reconstruction of the subject's anatomy that is based on the first medical imaging data and the second medical imaging data.

2. The computer-implemented method of claim 1, wherein updating the medical imaging reconstruction of the subject's anatomy includes:
causing the at least one of the first medical imaging device or the second medical imaging device to acquire first or second medical imaging data, respectively, from at least one region of the subject's anatomy such that the acquisition causes a reduction in the divergence; and
modifying the medical imaging reconstruction of the subject's anatomy based on the acquired first or second medical imaging data.

3. The computer-implemented method of claim 2, wherein causing the at least one of the first medical imaging device or the second medical imaging device to reacquire first or second medical imaging data, respectively, includes operating an actuator configured to one or more of reposition or reorient the at least one of the first medical imaging device or the second medical imaging device so that the at least one of the first medical imaging device or the second medical imaging device is targeting the at least one region of the subject's anatomy.

4. The computer-implemented method of claim 3, wherein:
the at least one region of the subject's anatomy includes a plurality of regions; and
the actuator is operated so that the first or second medical imaging data is reacquired for the plurality of regions according to one or more of a random or pseudo-random pattern, a linear pattern, a tilt pattern, a rotational pattern, or an obstacle-avoidance protocol based on a signal from a force transducer associated with the actuator.

5. The computer-implemented method of claim 2, wherein:
the at least one of the first medical imaging device or the second medical imaging device that is operable to selectively target different regions of the subject's anatomy includes a plurality of sensors or sensor arrays, each of the plurality of sensors or sensor arrays one or more of positions or oriented so as to target different regions of the subject's anatomy; and
causing the at least one of the first medical imaging device and the second medical imaging device to reacquire first or second medical imaging data, respectively, includes changing a targeting of the plurality of sensors or sensor arrays.

6. The computer-implemented method of claim 1, wherein the divergence in the first medical imaging data and the second medical imaging data is based on a distance between one or more of:
a first region of the subject's anatomy targeted by the first medical imaging device and a second region of the subject's anatomy targeted by the second medical imaging device; or
a first location of the first medical imaging device and a second location of the second medical imaging device.

7. The computer-implemented method of claim 6, wherein the divergence in the first medical imaging data and the second medical imaging data is based on a moving average of the distance.

8. The computer-implemented method of claim 6, further comprising one or more of:
determining one or more of the first location of the first medical imaging device or the first region of the subject's anatomy targeted by the first medical imaging device based on one or more of electromagnetic position tracking, fiber optic shape sensing, location tracking via the second medical imaging device, or impedance-based tracking: or
determining one or more of the second location of the second medical imaging device or the first region of the subject's anatomy targeted by the second medical imaging device based on one or more of electromagnetic position tracking, optical tracking, or internal position tracking of the second medical imaging device.

9. The computer-implemented method of claim 1, wherein the divergence in the first medical imaging data and the second medical imaging data is based on an extent of time elapsed since (a) one or more of the first medical imaging data was captured by the first medical imaging device or (b) the second medical imaging data was captured by the second medical imaging device.

10. The computer-implemented method of claim 1, further comprising:
generating the medical imaging reconstruction of the subject's anatomy based on the first medical imaging data and the second medical imaging data; and
causing a display to output a visual depiction of at least a portion of the medical imaging reconstruction.

11. The computer-implemented method of claim 10, wherein updating the medical imaging reconstruction of the subject's anatomy includes modifying the visual depiction of the at least portion of the medical imaging reconstruction such that at least a portion of the medical imaging reconstruction has a visual appearance indicative of an extent of time elapsed since medical imaging data upon which the at least portion is based was captured.

12. The computer-implemented method of claim 11, wherein the visual appearance indicative of an extent of time elapsed since medical imaging data upon which the at least portion is based was captured includes one or more of:
a reduction in opacity, contrast, saturation, sharpness, resolution, brightness, or combinations thereof, in which a magnitude of the reduction increases with an increase in the extent of time elapsed;
a shift in color, in which a magnitude of the shift increases with an increase in the extent of time elapsed;
a flashing effect; or
a bounding region or selection indicating the at least portion of the medical imaging reconstruction.

13. The computer-implemented method of claim 10, further comprising:
obtaining a prior medical imaging reconstruction of the subject's anatomy based on third medical imaging data captured by a third medical imaging device;
determining a registration of the medical imaging reconstruction with the prior medical imaging reconstruction; and
causing the display to output a visual depiction of the prior medical imaging reconstruction in conjunction with the at least portion of the medical imaging reconstruction based on the determined registration.

14. The computer-implemented method of claim 13, further comprising:
receiving a user selection for a relative visibility of the prior medical imaging reconstruction and the at least portion of the medical imaging reconstruction in the visual depiction; and
selectively increasing or decreasing a visibility of the prior medical imaging reconstruction and a visibility of the at least portion of the medical imaging reconstruction in the visual depiction, respectively, based on the user selection.

15. The computer-implemented method of claim 1, wherein the at least one first medical imaging device includes one or more of an ultrasound transducer, a parallel or phased array transducer, an optical coherence tomography device, or an optical sensor.

16. The computer-implemented method of claim 1, wherein the at least one second medical imaging device includes one or more of an ultrasound transducer, an array of ultrasound transducers, an X-ray device, or a computed tomography device.

17. A system for medical imaging reconstruction of a subject's anatomy, comprising:
a memory storing instructions;
a first medical imaging device configured to capture first medical imaging data from within the subject's anatomy;
a second medical imaging device configured to capture second medical imaging data from outside of the subject's anatomy, wherein at least one of the first medical imaging device or the second medical imaging device is operable to selectively target different regions of the subject's anatomy; and
a processor that is operatively connected to the memory, the first medical imaging device, and the second medical imaging device, and that is configured to execute the instructions to perform operations, including:
monitoring first medical imaging data of the subject's anatomy and second medical imaging data of the subject's anatomy; and
in response to detecting, based on the monitoring, a divergence in the first medical imaging data and the second medical imaging data above a predetermined threshold, updating a medical imaging reconstruction of the subject's anatomy that is based on the first medical imaging data and the second medical imaging data.

18. The system of claim 17, wherein updating the medical imaging reconstruction of the subject's anatomy includes:
causing the at least one of the first medical imaging device and the second medical imaging device to acquire first or second medical imaging data, respectively, from at least one region of the subject's anatomy that causes a reduction in the divergence; and
modifying the medical imaging reconstruction of the subject's anatomy based on the acquired first or second medical imaging data.

19. The system of claim 17, wherein the operations further include:
generating the medical imaging reconstruction of the subject's anatomy based on the first medical imaging data and the second medical imaging data; and
causing a display to output a visual depiction of at least a portion of the medical imaging reconstruction;
wherein updating the medical imaging reconstruction of the subject's anatomy includes modifying the visual depiction of the at least portion of the medical imaging reconstruction such that at least a portion of the medical imaging reconstruction has a visual appearance indicative of an extent of time elapsed since medical imaging data upon which the at least portion is based was captured.

20. A computer-implemented method for medical imaging reconstruction of a subject's anatomy, comprising:
monitoring first medical imaging data of the subject's anatomy and second medical imaging data of the subject's anatomy, wherein:
the first medical imaging data is captured from within the subject's anatomy by at least one first medical imaging device;
the second medical imaging data is captured from outside of the subject's anatomy by at least one second medical imaging device; and
at least one of the first medical imaging device or the second medical imaging device is operable to selectively target different regions of the subject's anatomy; and
in response to detecting, based on the monitoring, a divergence in the first medical imaging data and the second medical imaging data above a predetermined threshold, updating a medical imaging reconstruction of the subject's anatomy that is based on the first medical imaging data and the second medical imaging data, wherein updating the medical imaging reconstruction of the subject's anatomy includes:
causing the at least one of the first medical imaging device or the second medical imaging device to acquire first or second medical imaging data, respectively, from at least one region of the subject's anatomy such that the acquisition causes a reduction in the divergence;
modifying the medical imaging reconstruction of the subject's anatomy based on the acquired first or second medical imaging data; and
modifying a visual depiction of at least a portion of the medical imaging reconstruction such that the at least portion of the medical imaging reconstruction has a visual appearance indicative of an extent of time elapsed since medical imaging data upon which the at least portion is based was captured.
